(19) United States
(12) Patent Application Publication
(10) Pub. No.: US 2010/0249584 A1
(43) Pub. Date: Sep. 30, 2010

(54) VENTILATION APPARATUS FOR PULMONARY SCINTIGRAPHY

(75) Inventor: Roberto ALBERTELLI, Tirrenia (IT)

Correspondence Address: DENNISON, SCHULTZ & MACDONALD, 1727 KING STREET, SUITE 105, ALEXANDRIA, VA, 22314, US

(73) Assignee: AZIENDA OSPEDALIERO- UNIVERSITARIA PISANA, Pisa (IT)

(21) Appl. No.: 12/759,736

(22) Filed: Apr. 14, 2010

Related U.S. Application Data
(63) Continuation-in-part of application No. 10/508,015, filed on Sep. 29, 2004, now abandoned.

(30) Foreign Application Priority Data
Mar. 28, 2002 (IT) PI2002A000018

(51) Int. Cl. A61B 6/02; A61M 11/00

(52) U.S. Cl. 600/431; 128/200.23

(57) ABSTRACT

A ventilation apparatus (1) for pulmonary scintigraphy, having an inhalation collector (10) to which an element of inhalation (20), a feed duct (31), defining a flow of marked aerosol (55), and an outlet duct (32), for disposal of exhaled flow and any aerosol surplus, are connected. The feeding air may be of bi-level type, whereby the air flow rate when breathing out is low, even if always positive. The collector (10) has a first interposition element (21) located at the connection with the air feeding tube (31) that only allows inlet flow towards the patient. A second interposition element (22) is located at the connection with outlet duct (32). The presence of one-way valve(s) (21, 22) allows breathing of the marked aerosol in the bronchial branches of the patient (20) in a completely natural manner following physiologic respiration and avoiding hyper-accumulation of marker in the largest bronchial branches. Scintigraphy is also allowed during non-invasive mechanical ventilation, carried out through the ventilation apparatus (1).

This application is a continuation-in-part of Ser. No. 10/508,015, filed Sep. 29, 2004, which is a filing under 35 USC 371 of PCT/IB03/01156 filed Mar. 28, 2003.

The present invention relates to the medical field and in particular it relates to a ventilation apparatus for pulmonary scintigraphy, capable to provide a uniform distribution of a marking substance in a patient's lungs.

Furthermore, the invention relates to a method for inhalation of this substance.

### BACKGROUND OF THE INVENTION

As known, Lungs Ventilatory Scintigraphy is a technique for detecting the distribution of lungs ventilation in patients with pathology of the respiratory system such as bronchial asthma, chronic bronchitis and emphysema. It consists in ventilating into the patient an inert gas such as ¹³³Xe, ¹²⁷Xe, the ⁸¹Kr, or aerosol, usually ⁹⁹mTc-DTPA (diethylen-triamino-pentacetic acid) or sulphide colloidal ⁹⁹mTc, for then looking at its distribution in the lungs.

With the perfusion and ventilation systems presently in use, the patient breathes in marked aerosol, i.e. aerosol with dissolved radioactive material, through a mouthpiece connected on an end of a tube and connected at the other end to an aerosol apparatus, and breathes out the surplus marker through the same tube towards a radioactive material collecting filter. The air pressure is maintained steady when breathing in and out, so that the marker is deposited in the largest bronchial branches of the respiratory system. The radioactive material, therefore, deposits especially in the main bronchial branches, colliding against their walls, whereas it reaches the peripheral branches much slowly.

For taking a full picture with a “gamma camera” of the main and peripheral respiratory tubes the patient must ventilate them with a traditional aerosol apparatus for about 20/30 minutes. In fact, only such a long ventilation allows the radioactive substances to deposit also in the most peripheral tubes, in order to take an overall picture of the lungs.

On the other hand, for diffusing the radioactive material in the lungs, enough to provide readable distribution images of the ventilation (at least 1 Kc/sec), not only the above aerosol time is necessary (about 20/30 minutes) but also time is necessary for taking the images twice, i.e. preliminary images, immediately after counting 1 KC/sec, and deferred images, three/four hours later. This double test, in two different times, is necessary to avoid that the second image shows a radioactive accumulation in the main respiratory tubes. Such accumulation of radioactive material affects the acquisition of the first image, which since they are “unclean” cannot be read, unless comparing it with the second, for ascertaining possible pulmonary occlusions. In order to obtain “a clean image” it is necessary to wait for about four hours, so that the mucociliary system eliminates partially the accumulation of radioactive material. Notwithstanding the deferred images do not show said radioactive accumulation, however they cannot avoid showing the marker swallowed with the saliva through the oesophagus and the stomach, since scintigraphy detects it.

Another problem is that a scintigraphy test, during Non-Invasive Mechanical Ventilation with nasal mask, cannot be carried out without affecting the pressure control on which the apparatus is based and without polluting the apparatus and the environment with the exhaled marked aerosol.

It is desirable to improve the quality of marked air that the patient breathes in for the following reasons:
If the breathed in air is too rich of marker, the patient has to wait a long time before being exposed to scintigraphy, to expel the excess of marker. This occurs especially if the marking particles are too large.
If the breathed in air is too poor of marker, the patient has to breathe in and out the marked air for a long time.
In both cases, the patient is subject to a quantity of dangerous marking particles more than necessary. This occurs especially if the marking particles are too small.
In both cases, the marking particles deposit mostly in the first airways (mouth, throat and bronchial airways) and in the junctions between airways of the patient. The most remote parts of the lungs that are the most important for the scintigraphy test and are those where not much marking particles deposit, causing the scintigraphic imaging to be worse in that parts. For this reason it is necessary a long breathing time (up to ½ hour) and then a long additional time to expel the excess of marker accumulated in the junctions, up to reaching a condition where the scintigraphy imaging can be interpreted for a diagnosis on presence of obstructions in the lungs.

U.S. Pat. No. 4,803,977 provides an inhalation collector, where a fresh marked aerosol enters and a breathed out mixture of marked aerosol exits. Check valves are present both at the inlet and at the outlet of the inhalation collector. The inhalation collector is far from the mouth of the patient with a long portion of duct in common for the breathed in and breathed out flows. This interference is sought by Kremer such that the size of marked particles that reach the patients are less than 1 μm.

### SUMMARY OF THE INVENTION

It is therefore object of the present invention to provide a ventilation apparatus for pulmonary scintigraphy that is able to select the size of particles of marked aerosol that reaches the patient and therefore to improve the quality of marked air that the patient breathes in.

It is another object of the present invention to provide a ventilation apparatus that provides a uniform distribution of the marked aerosol in the bronchial branches, i.e. without hyper accumulation of marker, reducing the impact on the bronchial branches and making easier a direct peripheral distribution of the marker.

It is another object of the present invention to provide a ventilation apparatus for carrying out pulmonary scintigraphy even on not cooperating patients.

It is also object of the present invention to provide a ventilation apparatus for pulmonary scintigraphy capable of reducing the time necessary for executing a scintigraphical test.

It is a further object of the present invention to provide a ventilation apparatus for pulmonary scintigraphy for carrying out at the same time a non invasive mechanical ventilation with nasal mask, without affecting the pressure cycles on which the apparatus works and without polluting the apparatus and the environment with exhaled marked aerosol.

These and other objects are achieved by the ventilation apparatus for pulmonary scintigraphy, according to the present invention, comprising:
a source of marked aerosol;
an element of inhalation, through nose or mouth that is adapted to administer to a patient said marked aerosol;
a feed duct that is connected to said source of marked aerosol in order to define an inlet flow of marked aerosol towards said element of inhalation;
an outlet duct in order to define an outlet flow of exhaled/surplus aerosol from said element of inhalation;
an inhalation collector where said feed duct, said outlet duct and said element of inhalation converge, wherein said element of inhalation is connected directly to said inhalation collector;
a one-way interposition element in said inhalation collector that is movable between a first position, where it allows said inlet flow of marked aerosol when breathing in from said element of inhalation, and a second position, where it stops or limits the flow into said inhalation collector of the marked outlet aerosol when breathing out from said element of inhalation towards said outlet duct;

wherein said one-way interposition element is a flap valve, and

wherein said feed duct has a predetermined length set between 20 cm and 40 cm that is adapted to retain particles of marked aerosol that are larger than 1.5 μm and to let particles of marked aerosol that have a size set between 1.0 μm and 1.5 μm to pass through said feed duct and to reach said element of inhalation when breathing in.

Advantageously, the feed duct has a predetermined length set between 25 cm and 35 cm.

Preferably, the feed duct has a predetermined diameter set between 2 cm and 3 cm, for example 2.5 cm.

In particular, the feed duct can be a corrugated flexible duct.

In particular, the source of marked aerosol comprises a Venturi jet nebulizer adapted to be fed by an air flow set between 15-25 l/min and to generate a flow of marked aerosol including marked aerosolized particles.

Advantageously, the nebulizer is fed by an air flow set between 18-20 l/min.

Advantageously, the air marked flow is of bi-level type, such that an air flow rate when breathing out is positive and lower than an air flow rate when breathing in.

In particular, the feed duct has a volume set between 100 cm³ and 300 cm³.

Advantageously, the feed duct has a volume set between 150 cm³ and 250 cm³.

The inhalation collector is located very close to the zone of inhalation, either through nose or mouth in order to assure that the inlet marked aerosol flows through a way different from the outlet marked aerosol.

In particular, the flap valve comprises a stiff plastic foil that is elastically flexible.

Advantageously, the flap valve is adapted to assume intermediate positions between said first and second position to follow a demand of air of the patient. More in detail, in addition to an open and a closed position, the flap valve can assume also intermediate positions in order to follow the breathing demand of the patient. In fact, when breathing in and out a gradient of pressure is produced in the inhalation collector suitable for creating a flow of marked aerosol following the physiological respiration of the patient.

The foil deflects only in one direction owing to an annular abutment, added to or made in the collector or in the tube, on which the foil same is connected at a side and on which it rests when in closed position. Then, each foil has a fixed portion, integral to the inner collector wall, about which the other portion of the foil can rotate between the first and the second position. The annular abutment is co-axial to the portion of the collector, or of the duct in which it is arranged, and has inner diameter smaller than that of the respective foil.

Advantageously, the flow of marked aerosol coming from a nebulizer is led into the air feed duct upstream of the collector.

Alternatively, the flow of marked aerosol coming from a nebulizer is led directly into the collector through a duct different from that of the air feed duct.

In a first embodiment of the invention, the inhalation collector is a three-way collector, with a central chamber and three apertures. In particular, the following are provided:
a first opening for connecting said central chamber with said feed duct;
a second opening for connecting the central chamber with said outlet duct which leads to a radioactive material collecting filter;
a third opening for connecting the central chamber with said element of inhalation;
a second flap one-way valve at the second opening.

More in detail, when the patient breathes in the marked aerosol the flap valve at the first opening opens completely, whereas the valve at the second opening is completely closed. When breathing out, instead, the first valve blocks partially or completely the relative aperture responsive to local pressures, reducing the flow of the aerosol, already at a low level in a bi-level ventilation, whereas the second valve opens and allows to dispose of the exhaled flow, thus following the patient's respiration.

In other words, the first valve reduces the inlet flow and allows it completely when breathing in. This creates a vortex in the large bronchial branches, without allowing the radioactive material to deposit by a stirring action. When breathing out, the surplus radioactive material is eliminated, leaving in the lungs only that necessary for marking. This way, the bronchial branches can be marked in a few minutes (from about 2 to 6 minutes) up to the final marking, and in this time it is possible to take the radiographs for looking at the transient phases.

In a second embodiment of the invention, the inhalation collector is a four-way collector, with a central chamber and four apertures. In particular, the following are provided:
a first opening for connecting said central chamber to said feed duct;
a second opening for connecting the central chamber with said outlet duct which is connected to a radioactive material collecting filter;
a third opening for connecting the central chamber with a feed duct of air for non invasive mechanical ventilation;
a fourth opening for connecting the central chamber with said element of inhalation.

### BRIEF DESCRIPTION OF THE DRAWINGS

Further characteristics and advantages of the present invention, will be made clearer with the following description of possible embodiments, exemplifying but not limitative, with reference to the attached drawings, wherein:

FIG. 1 shows diagrammatically a partially cross sectioned elevational side view of a first embodiment of a mouth ventilation apparatus for pulmonary scintigraphy;

FIG. 1A is a enlarged view of a detail of FIG. 1 for showing the mechanism of operation of a possible embodiment of a one-way interposition element;

FIG. 2 shows diagrammatically a perspective view of the apparatus of FIG. 1 applied to a patient;

FIG. 3 shows a perspective view of a first embodiment of an inhalation collector used in the apparatus of FIG. 1;

FIG. 4 shows the collector of FIG. 3 in a cross sectional view that shows the relative positions of the different interposition elements when breathing in;

FIG. 4A shows diagrammatically the pressure trend versus time in the apparatus of FIG. 1 when breathing in;

FIG. 5 shows the collector of FIG. 3 in a cross sectional view that shows the relative positions of the different interposition elements when breathing out;

FIG. 5A shows diagrammatically the pressure trend versus time in apparatus of FIG. 1 when breathing out;

FIG. 6 shows diagrammatically the pressure trend versus time in apparatus of FIG. 1 during a full cycle of breathing in and out;

FIG. 7 shows diagrammatically a partially cross sectioned elevational front view of a ventilation apparatus for pulmonary scintigraphy according to an embodiment alternative to that of FIG. 1 with nasal ventilation;

FIG. 8 shows a perspective view of the inhalation collector used in the apparatus of FIG. 7;

FIG. 9 shows diagrammatically a perspective view of the location of different components in the apparatus of FIG. 7, relatively to a patient when breathing in;

FIG. 10 is a partially cross sectioned elevational side view of a ventilation apparatus for pulmonary scintigraphy according to another embodiment during inhalation of marked aerosol;

FIG. 11 shows the embodiment of the ventilation apparatus for pulmonary scintigraphy of FIG. 10 during exhalation of surplus aerosol.

### DESCRIPTION OF A PREFERRED EMBODIMENT

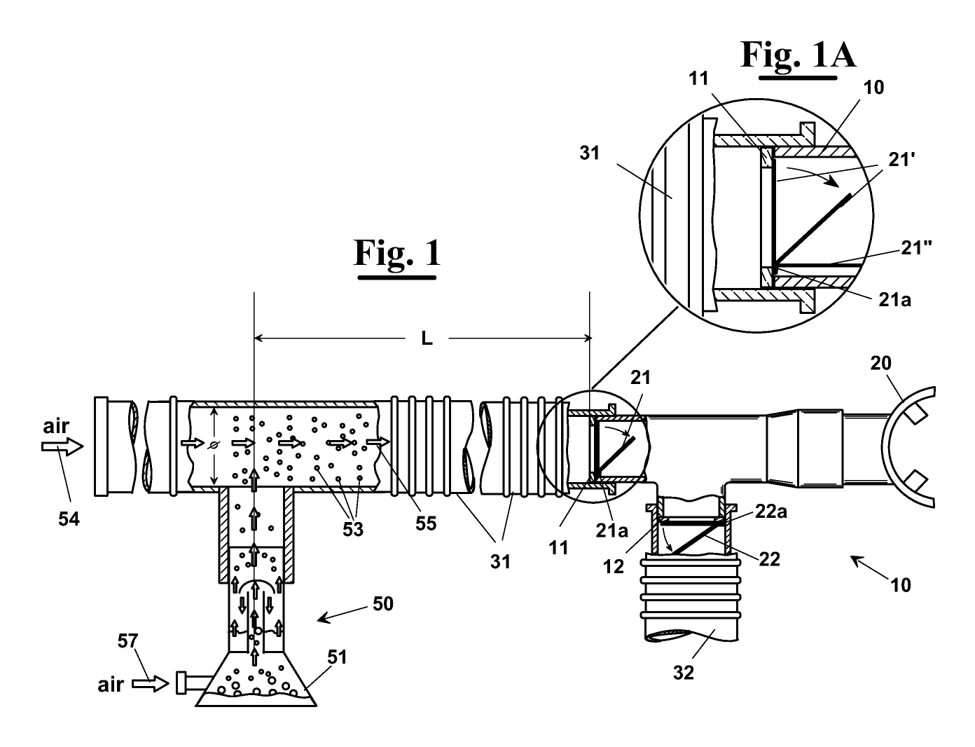

In FIG. 1 a first embodiment is shown of a ventilation apparatus 1 for pulmonary scintigraphy, according to the present invention.

It comprises an inhalation collector 10 to which an element of inhalation, for example a mouthpiece 20, a feed duct 31, defining a flow of marked aerosol 55, and an outlet duct 32 for disposal of exhaled flow 56 and any aerosol surplus, are connected. Normally the feeding air is of bi-level type, whereby the air flow rate when breathing out is low, even if always positive.

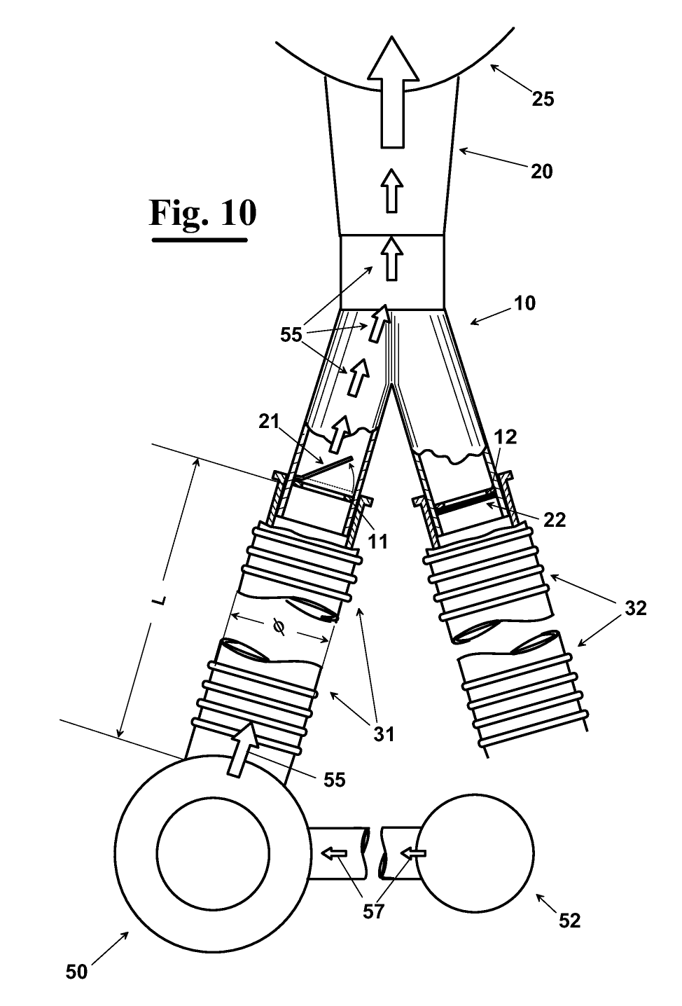

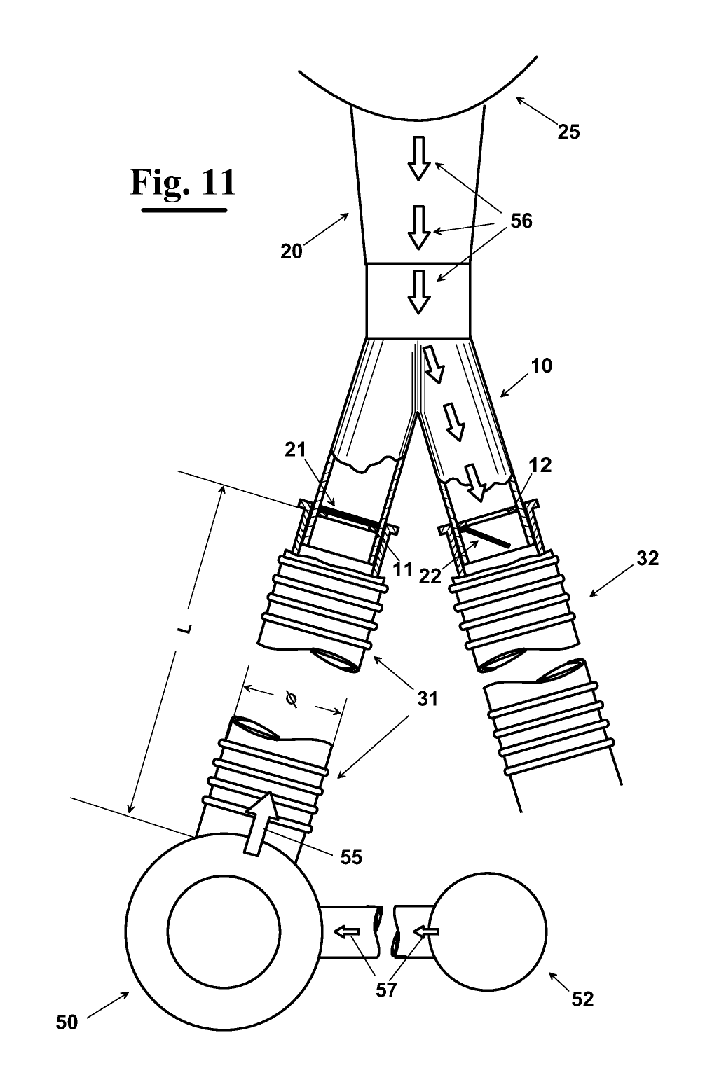

In particular, as shown in FIG. 1, the flow of marked aerosol 55 is obtained mixing upstream of collector a current of nano-colloidal particles 53, marked for example with Tc-99m and delivered by a nebulizer 50, with an air flow 54. In particular, nebulizer 50 is a Venturi jet nebulizer adapted to be fed by an air flow 57 set between 15-25 l/min and to generate a flow of marked aerosol 55 including marked aerosolized particles 53, i.e. marked droplets, sized between 0.01 μm and 7 μm. Preferably, nebulizer 50 is fed by an air flow rate 57 set between 18-20 l/min, that can be generated by an air pump 52 (FIGS. 10, 11) or by a compressor (not shown) upstream of nebulizer 50.

As shown in FIG. 1 and in detail in FIG. 1A, collector 10 has, at the connections with ducts 31 and 32, one-way interposition elements 21 and 22. In particular, a first interposition element 21 is located at the connection with air feeding tube 31 and only allows inlet flow towards the patient, whereas a second interposition element 22 is located at the connection with outlet duct 32.

In the case shown in figures from 1 to 5, each one-way interposition element 21 or 22, is a flap valve formed by a foil of stiff thin plastic material capable of bending elastically associated to a ring abutment 11 or 12, solid or added, co-axial to the respective portion of collector 10 and having inner diameter smaller than the width of the respective valve. Each foil of plastic material 21 has a fixed portion 21a, integral to the inner wall of collector 10, about which the other portion of foil 21 can rotate between a first position 21′, where it stops or limits the flow 55 of marked aerosol, and a position 21″, wherein a maximum cross section is offered to the flow of marked aerosol towards collector 10 (FIG. 1A). Valve 22 is made in a similar way for a flow exiting from collector 10. In the closed position elements 21 and 22 rest on rings 11 and 12.

The presence of the one-way valves 21 and 22 allows breathing the marked aerosol in the bronchial branches of the patient 25 in a completely natural manner following the physiologic respiration and avoiding hyper-accumulation of marker in the largest bronchial branches.

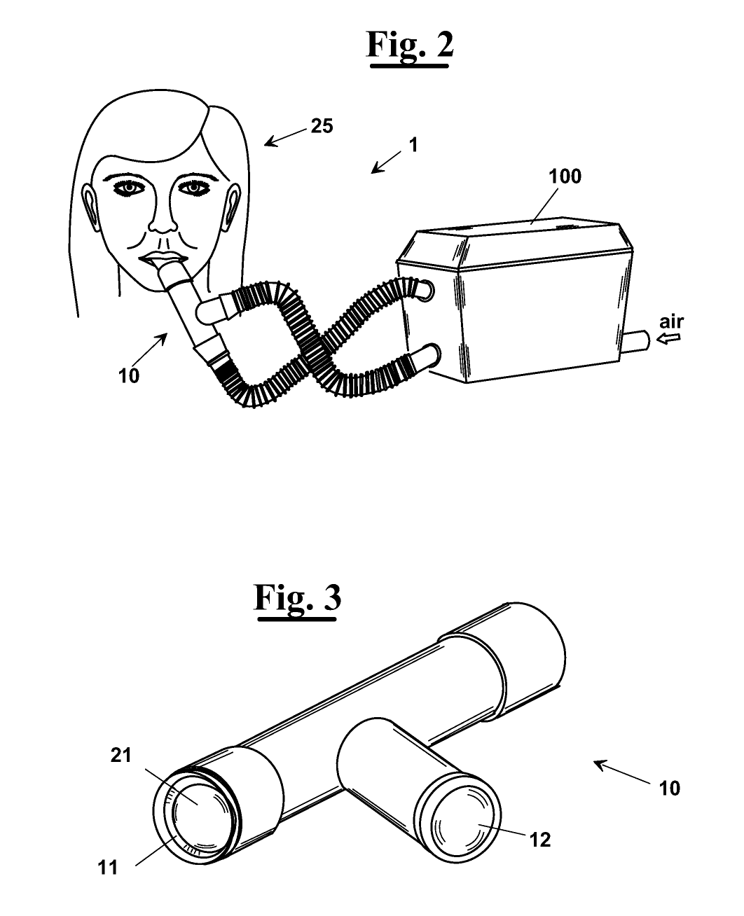

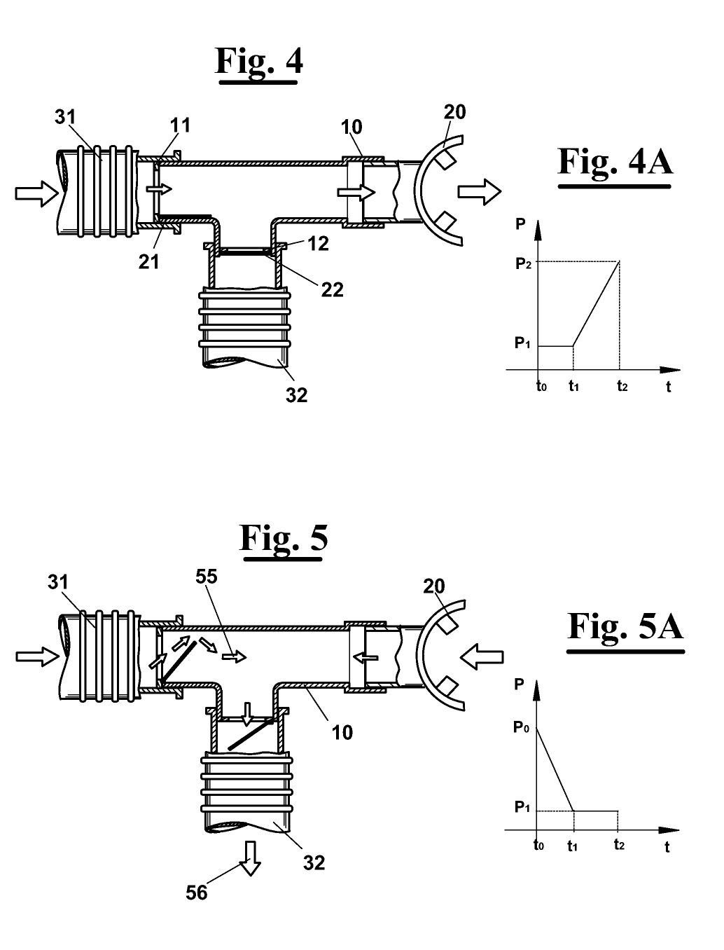

The device can be used, for example, as shown in FIG. 2, with respective ducts 31, 32 and mouthpiece 20 connected to collector 10 of FIG. 3. When breathing in, the pressure of the aerosol is maximum and the valve 21 is completely open (FIG. 4).

As shown both in FIGS. 1 and 10, according to the invention, feed duct 31 has a predetermined length “L” set between 20 cm and 40 cm. This particular length of duct 31 is selected in order to retain particles of marked aerosol that are larger than 1.5 μm and to let particles of marked aerosol that have a size set between 1.0 μm and 1.5 μm to pass through said feed duct 31 and to reach the element of inhalation when breathing in. Advantageously, feed duct 31 has a predetermined length L set between 25 cm and 35 cm, and in a particularly preferred embodiment feed duct 31 has a length of 30 cm.

Preferably, feed duct 31 has a predetermined diameter Ø set between 2 cm and 3 cm, and in a particularly preferred embodiment feed duct 31 has a diameter of 2.5 cm and is a corrugated flexible duct. This way, the volume of feed duct is comprised between 100 cm³ and 300 cm³, preferably between 15 cm³ and 250 cm³.

According to the invention air and marker 51 are mixed in 50 upstream of collector 10 and proceed through feed duct 31. Marked flow 55 is very unstable, because the marked particles 53 are droplets that deposit very easily on the surfaces of the ducts that they meet. This occurs not only in the airways of the patient's lungs, but also in the feed duct 31 and in the inhalation collector 10. As the flow of air stops, owing to the breathed out stream that tends to close flap valve 21, the pressure increases in feed duct 31 and larger particles 53 deposit on feed tube 31 inner walls. Larger particles 53 deposit in a faster way than smaller ones.

In particular, particles of marked aerosol that are larger than 1.5 μm fall within feed duct 31 and are retained by its walls, owing to its optimum length, whereas let particles of marked aerosol 55 that have a size set between 1.0 μm and 1.5 μm pass through feed duct and reach inhalation collector 10. This occurs continuously, because there is a continuous supply of air. The patient breathes in and out, and there is a peak of pressure just after the beginning of the breathing out phase that closes flap valve 21. Then the pressure decreases during the breathing out phase (FIGS. 4 and 11).

By using flap valve 21 for the inlet port 11 of inhalation collector 10, there is a surprising and unexpected effect that flap valve 21 smoothly opens and closes, and in particular it opens up to an intermediate position when the patient still is breathing out, in the last part of the breathing out cycle when pressure is decreasing, owing to the positive pressure generated by the air feed flow. The use of a flap valve 21 at inlet port 11 does not block abruptly and for a long time the flow coming from tube 31 of air and marker, but smoothly and for a short time, only during the pressure peak of the breathing out cycle that causes a corresponding pressure peak in feed duct 31. Flap valve 21 opens again in the final part of the breathing out cycle, owing to the raising pressure in feed duct and decreasing pressure of the breathing out step.

This cyclical and not abrupt movement of flap valve 21 cooperates with feed duct 31 to select the desired size of marked droplets/particles between 1.0 μm and 1.5 μm is not retained by feed duct 31, whereas larger particles are blocked. Of course, smaller particles than 1.0 μm pass, but they are less relevant for a quick and effective marking of the lungs, and they are easily breathed out also.

In this way, the quality of the marker is surprisingly improved, and the size of marking particles is ideal for scintigraphy, because they reach all the parts of the lungs, without concentrating too much in the larger airways and at the junctions of the lungs, and reach the most inner airways. In a short time the patient is ready for scintigraphy, which is of a very good quality.

Other valves such as check valves do not have this characteristic: they close abruptly and open abruptly only when the breathing in cycle starts again. If a check valve were used instead of a flap valve 21, it would stay closed for a too long time, and much of the particles would deposit in tube 31, with too small particles like in Kremer (less than 1 μm) reaching the lungs.

For this reason, flap valve 21 has not only the function of providing a unidirectional flow in the inlet duct, but also is a member for selecting the quality of marked particles entering the inhalation collector.

It is also important that the quality of marker is not disturbed as far as possible by the distance between the inhalation collector and the patient, and this is achieved by connecting the inhalation collector directly to the mouth, by mouthpiece 20. The anticipated opening movement of flap valve 21 causes a slight overpressure in inhalation collector 10, and when the patient starts breathing in again, there is a ready turbulent flow of air and marker, that prevents the particles to deposit in the first airways, reaching quickly the remote airways of the lungs.

When breathing out, on valve 21 a certain pressure is made by the flow 56 exhaled by patient 25 in a direction opposite to the even low positive pressure of the fed aerosol flow, but of lower intensity. This causes valve 21 to move to an intermediate position that reduces but does not stop the aerosol flow into collector 10. Therefore, in collector 10 the flow of exhaled air and the surplus aerosol flow sum to each other increasing the pressure inside. This causes valve 22 to open for disposing of the surplus flow exhaled by patient 25 (FIG. 5). Valve 21, that reduces the aerosol flow towards collector 10 when breathing out and leaves it free when breathing in, when opens again produces a pressure variation that creates a vortex in the large bronchial branches of the patient without allowing the radioactive material 53 to deposit, owing to a stirring action of the vortex. When breathing out the radioactive material is eliminated along with the surplus air. This way the bronchial branches can be marked in a few minutes (from about 2 to 6 minutes).

Therefore, the invention is the original combination of the use of a feed duct of determined length

The use of a flap valve 21 as a unidirectional valve at the inlet 11 in inhalation collector 10, which it is not only a unidirectional flow member, but it is also a member for selecting the quality of marked particles, and

the mouthpiece 20 directly connected to inhalation collector 10, to limit a deposit of the marking particles in the first airways of the patient.

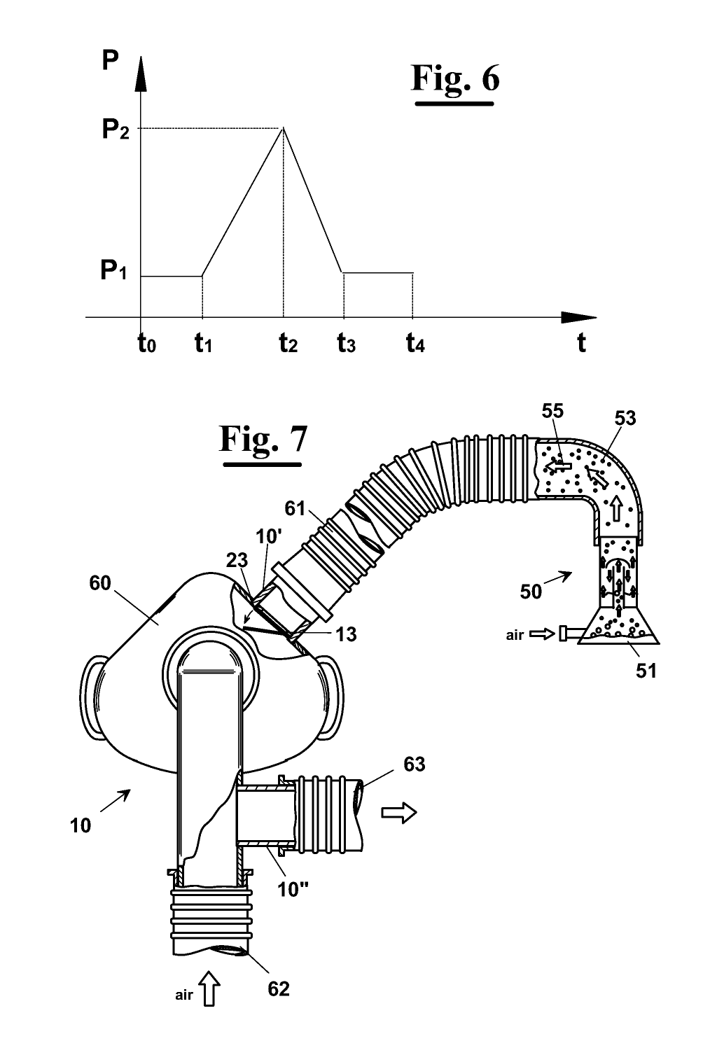

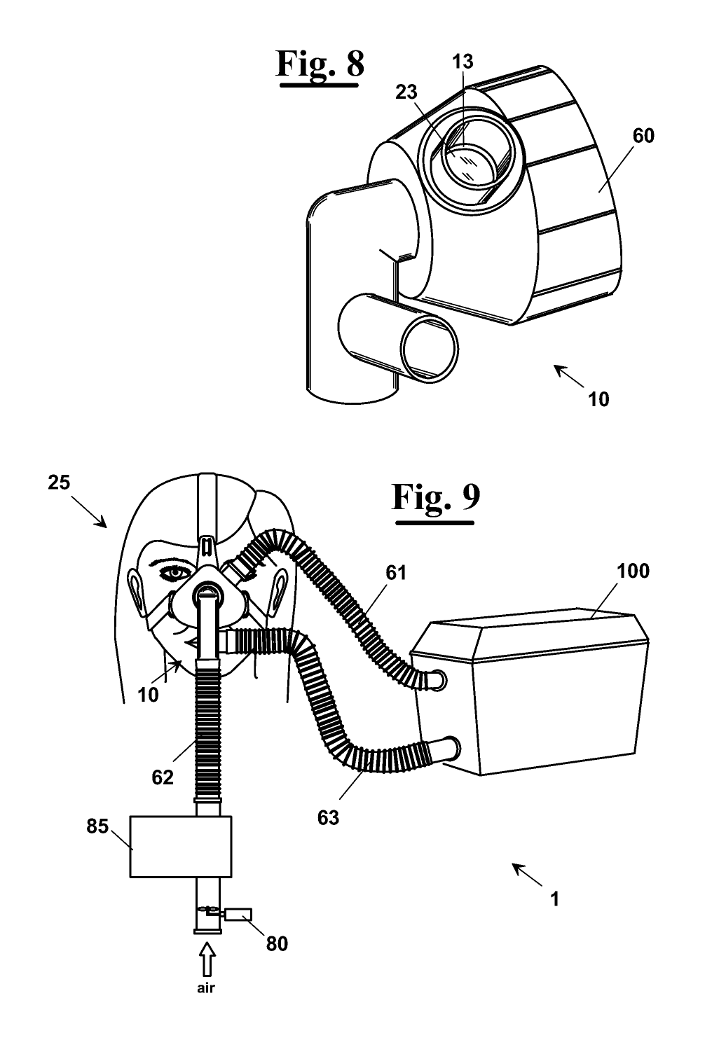

What above said is graphically shown in FIGS. 4A, 5A and 6 where qualitative trends of pressure (P) versus time (t) are indicated respectively when breathing in, when breathing out and during a full cycle of breathing in and out. In particular, when breathing in the pressure rises from a starting value P₁ up a maximum value P₂, whereas when breathing out the pressure decreases from a starting value P₀ to a value P₁<P₀, enough to keep valve 21 half open.

In figures from 7 to 9 an alternative embodiment is shown of a pulmonary ventilation scintigraphy apparatus alternative to that shown in figures from 1 to 5. This embodiment has the advantage of carrying out a pulmonary scintigraphy during a Non Invasive Mechanical Ventilation, or NIMV, without affecting the pressure cycles on which apparatus 1 works and without polluting apparatus 1 and the environment with exhaled marked aerosol. As shown in FIG. 7, a collector 10 integrated in a nasal mask 60 of an apparatus for mechanical ventilation is normally connected to a primer 80 (FIG. 9) that produces a flow of air towards the mask same.

For carrying out a pulmonary scintigraphy, using a similar apparatus, in nasal mask 60 a one-way valve 23 is arranged allowing only an inlet flow, like that above described (FIG. 1A). Valve 23 is located in collector 10 at a connection 10′ with a feed duct 61 of an aerosol flow 55 produced by a nebulizer 50 as disclosed above.

Nasal mask 60 is, furthermore, connected by a connection 10″ of collector 10 to an outlet duct 63 of a exhalation flow containing the surplus marked aerosol, connected at the other end to a collecting filter, not shown, inserted into a lead walled box 100, in order to prevent scattering of radioactive material in the environment and to protect the operator.

During NIMV, at the moment of executing the test, it is sufficient to connect the feeding tube 61 to collector 10. In particular, nebulizer 50 works with a pressure sufficient to open valve 23 only when in the mask a vacuum is created caused by the inspiration of the patient, whereas it stops when breathing out. Differently from the previous case, the system mixes the air supplied by primer 80 and aerosol flow 55 of nano-colloidal particles 53 marked with Tc-99m coming from nebulizer 50 only in collector 10 and not before; therefore, the flow of marked aerosol to the patient 25 is adjusted by primer 80. This way, it is possible to measure the distribution of air supplied by primer 80 in the lungs of patient 25 thus allowing, with the aid of an imaging system, to check in real time the lungs apex-base ratio U/L.

The foregoing description of a specific embodiment will so fully reveal the invention according to the conceptual point of view, so that others, by applying current knowledge, will be able to modify and/or adapt for various applications such an embodiment without further research and without parting from the invention, and it is therefore to be understood that such adaptations and modifications will have to be considered as equivalent to the specific embodiment. The means and the materials to realise the different functions described herein could have a different nature without, for this reason, departing from the field of the invention. It is to be understood that the phraseology or terminology employed herein is for the purpose of description and not of limitation.

What is claimed is:

1. Ventilation apparatus for pulmonary scintigraphy comprising:
a source of marked aerosol;
an element of inhalation, through nose or mouth that is adapted to administer to a patient said marked aerosol;
a feed duct that is connected to said source of marked aerosol in order to define an inlet flow of marked aerosol towards said element of inhalation;
an outlet duct in order to define an outlet flow of exhaled/surplus aerosol from said element of inhalation;
an inhalation collector where said feed duct, said outlet duct and said element of inhalation converge, wherein said element of inhalation is connected directly to said inhalation collector
a one-way interposition element in said inhalation collector that is movable between a first position, where it allows said inlet flow of marked aerosol when breathing in from said element of inhalation, and a second position, where it stops or limits the flow into said inhalation collector of the marked outlet aerosol when breathing out from said element of inhalation towards said outlet duct,
wherein said one-way interposition element is a flap valve, and
wherein said feed duct has a predetermined length set between 20 cm and 40 cm that is adapted to retain particles of marked aerosol that are larger than 1.5 μm and to let particles of marked aerosol that have a size set between 1.0 μm and 1.5 μm to pass through said feed duct and to reach said element of inhalation during a breathing in step.

2. Apparatus according to claim 1, wherein said feed duct has a predetermined length set between 25 cm and 35 cm.

3. Apparatus according to claim 1, wherein said feed duct has a predetermined diameter set between 2 cm and 3 cm.

4. Apparatus according to claim 1, wherein said feed duct is a corrugated flexible duct.

5. Apparatus according to claim 1, wherein said source of marked aerosol comprises a Venturi jet nebulizer adapted to be fed by an air flow set between 15-25 l/min and to generate a flow of marked aerosol including marked aerosolized particles.

6. Apparatus according to claim 5, wherein said Venturi jet nebulizer is fed by an air flow set between 18-20 l/min.

7. Apparatus according to claim 5, wherein said air marked flow is of bi-level type, such that an air flow rate when breathing out is positive and lower than an air flow rate when breathing in.

8. Apparatus according to claim 1, wherein said feed duct has a volume set between 100 cm³ and 300 cm³.

9. Apparatus according to claim 8, wherein said feed duct has a volume set between 150 cm³ and 250 cm³.

10. Apparatus according to claim 1, wherein said flap valve comprises a stiff plastic foil that is elastically flexible and is adapted to assume intermediate positions between said first and second position to follow a demand of air of the patient.

11. Apparatus according to claim 10, wherein said flap deflects only one-way by an annular abutment to which said foil is connected at a side and on which it rests when in closed position.

12. Apparatus according to claim 1, wherein said inhalation collector is a three-way collector and has a central chamber with three apertures and precisely:
a first opening for connecting said central chamber with said feed duct;
a second opening for connecting the central chamber with said outlet duct which leads to a radioactive material collecting filter;
a third opening for connecting the central chamber with said element of inhalation;
a second flap one-way valve at the second opening.

13. Apparatus according to claim 1, wherein said inhalation collector is a four-way collector and has a central chamber with four apertures and precisely:
a first opening for connecting said central chamber to said feed duct;
a second opening for connecting the central chamber with said outlet duct which is connected to a radioactive material collecting filter;
a third opening for connecting the central chamber with a feed duct of air for non invasive mechanical ventilation;
a fourth opening for connecting the central chamber with said element of inhalation.

14. Inhalation collector suitable for being used in a ventilation apparatus for pulmonary scintigraphy according to claim 1.

15. Disposable kit comprising a collector, an inhalation element of through nose or mouth, ducts for inlet and outlet flows into/from said collector, suitable for being used in a ventilation apparatus for pulmonary scintigraphy according to claim 1.
